(19) United States
(12) Patent Application Publication
(10) Pub. No.: US 2026/0068910 A1
(43) Pub. Date: Mar. 12, 2026

(54) USES OF ASPERGILLUS NIGER FRUCTOSYLTRANSFERASE

(71) Applicant: AB Enzymes GmbH, Darmstadt (DE)

(72) Inventors: Klaudija MILOS, Darmstadt (DE); Andrzej DADAS, Darmstadt (DE); Volker MARSCHNER, Darmstadt (DE); Judit ZDIARSTEK, Darmstadt (DE); Felix SCHOLTEN, Darmstadt (DE); Timo STRESSLER, Darmstadt (DE)

(73) Assignee: AB Enzymes GmbH, Darmstadt (DE)

(21) Appl. No.: 19/107,467

(22) PCT Filed: Aug. 29, 2023
(86) PCT No.: PCT/EP2023/073614

§ 371 Date: Feb. 27, 2025

(30) Foreign Application Priority Data
Aug. 30, 2022 (EP) 22192972.2

(51) Int. Cl. A23L 2/02; A23L 2/84; C12N 9/10; C12N 9/26; C12N 9/42; C12N 9/88

(52) U.S. Cl. CPC A23L 2/02; A23L 2/84; C12N 9/1051; C12N 9/2411; C12N 9/2437; C12N 9/88; C12Y 204/01009; C12Y 302/01004; C12Y 402/0201

(57) ABSTRACT

The present invention relates to a method for converting sucrose comprised in a liquid food product into at least one fructooligosaccharide (FOS), the method comprising contacting said liquid food product with an Aspergillus niger fructosyltransferase under conditions allowing enzymatic conversion of sucrose to at least one FOS by said fructosyltransferase, wherein said contacting is performed at least until the sucrose concentration in said liquid food product is at most 15 g/L; and to compositions, uses, and products related thereto.

The present invention relates to a method for converting sucrose comprised in a liquid food product into at least one fructooligosaccharide (FOS), the method comprising contacting said liquid food product with an Aspergillus niger fructosyltransferase under conditions allowing enzymatic conversion of sucrose to at least one FOS by said fructosyltransferase, wherein said contacting is performed at least until the sucrose concentration in said liquid food product is at most 15 g/L; and to compositions, uses, and products related thereto.

Many food products produced from fruits or legumes contain high concentrations of sucrose. For many applications, reduction of sucrose concentration in such food products may be desirable. For this purpose, invertases have long been used, which hydrolyze sucrose into its monomeric sugar units, glucose and fructose. One member of the invertase family of enzymes is Aspergillus niger invertase, which was described by Yuan et al. (2006), Microbiology 152:3061 and in Boddy et al. (1993), Curr Genet 24: 60.

However, in particular under health perspectives, it is generally considered desirable to reduce the amounts of mono- and disaccharides in food products, e.g. to reduce their glycemic index. One option to achieve this goal is the use of fructosyltransferases, i.e. enzymes which transfer the fructose units of sucrose to sucrose or other acceptor saccharides, thereby generating fructooligosaccharides comprising several fructose moieties. Corresponding enzymes and methods have been described e.g. in EP 3 231 293 A1, EP 2 294 093 A1, EP 1951 885 A1, and WO 2008/102336 A2.

Notably, fructosyltransferases frequently also have invertase activity, in particular at low acceptor concentrations; i.e. with most fructosyltransferases, hydrolysis of sucrose is favored over transfer of fructose units already at relatively high sucrose concentrations. E.g. in EP 1 951 885 A1, using an Aspergillus japonicus fructosyltransferase, it was found that below sucrose concentrations of approx. 5%, i.e. 50 g/L, fructooligosaccharide production essentially stopped.

In view of the above, there is a need for improved means and methods for reducing sucrose content in food products. The technical problem underlying the present invention may be seen as the provision of means and methods for complying with the aforementioned need. The technical problem is solved by the embodiments characterized in the claims and herein below.

In accordance, the present invention relates to a method for converting sucrose comprised in a liquid food product into at least one fructooligosaccharide (FOS), the method comprising contacting said food product with an Aspergillus niger fructosyltransferase under conditions allowing enzymatic conversion of sucrose to at least one FOS by said fructosyltransferase.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof, thus, e.g. identifying “a cell” relates to identifying at least one cell, preferably to identifying a multitude of cells. In concurrence, reference to “at least one fructooligosaccharide” relates to at least one type of fructooligosaccharide, as specified herein below.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above. Also, the methods may be performed at any scale deemed appropriate by the skilled person, e.g. laboratory scale, test plant scale, or industrial scale.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (e.g. BLAST, GAP, BESTFIT, PASTA, or TFASTA), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. More preferably, the Basic Local Alignment Search Tool (BLAST) implementation is used with default parameter values for alignment. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, a fragment of a fructosyltransferase may in particular a sub-part of a fructosyltransferase described herein below still having the activity as described. Unless specifically indicated otherwise herein, the compounds specified, in particular the polynucleotides and polypeptides, may be comprised in larger structures, e.g. may be covalently or non-covalently linked to further sequences, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. Preferably, however, the polypeptides consist of the sequence specifically indicated.

The term “polypeptide”, as used herein, refers to a molecule consisting of several, typically at least 20 amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 75 to 1000, still more preferably of from 100 to 500, most preferably of from 110 to 400 amino acids. Preferably, the polypeptide is comprised in a fusion polypeptide and/or a polypeptide complex.

The term “sucrose” is understood by the skilled person to relate to β-D-Fructofuranosyl α-D-glucopyranoside, CAS No. 57-50-1, which is the major constituent of e.g. beet sugar and cane sugar.

The term “oligosaccharide” is understood by the skilled person. Preferably, an oligosaccharide is an oligomer comprising at most ten, preferably at most six, even more preferably at most five, still more preferably at most four, monosaccharide units. Also preferably, an oligosaccharide comprises at least three monosaccharide units, i.e. is a trisaccharide, or comprises at least four monosaccharide units. Thus, preferably, the oligosaccharide comprises of from 3 to 10, preferably of from 3 to 5, monosaccharide units.

As used herein, the term “fructooligosaccharide”, which may also be referred to as “FOS” or “oligofructan” herein, relates to an oligosaccharide as specified herein above comprising at least two fructose monomers and, optionally, at least one glucose monomer. Preferably, the FOS comprises one molecule of D-glucose in the terminal position and from 2 to 4 D-fructose units, wherein the linkages between fructose residues preferably are beta-(2-1) glycosidic linkages. Thus, preferably, an FOS is produced by transfer of one or more fructose units to sucrose. Preferably, the fructooligosaccharide comprises, preferably is, a Kestose, preferably 1-Kestose (CAS No. 470-69-9), Kestotetraose (preferably PubChem CID 91851626), and/or Kestopentaose (preferably PubChem CID 1337599).

The term “liquid food product” is understood by the skilled person. Preferably, the term “liquid” product, as used herein, includes all products which are not solid under standard conditions. Thus, liquid products are products assuming essentially the shape of the container they are comprised in, over at time frame of at most 1 hour. Thus, the term liquid food product also includes semisolid products such as e.g. purees. Thus, as used herein, the term “liquid food product” may also be replaced by the term “liquid or semi-solid food product”. Preferably, the viscosity of the liquid food product, preferably before being treated by the method of the present invention, is of from 1 mPa*s to 10000 mPa*s, more preferably of from 10 mPa*s to 6000 mPa*s, wherein said viscosity preferably is dynamic viscosity, preferably determined as described herein below in the Examples.

As used herein, the term “food product” relates to a composition of matter being safe for human consumption; preferably, suitability for human consumption is determined under applicable European Union and/or US regulations, preferably as of July 2022. Preferably, the term food product also includes compositions of matter intended and/or being safe for animal consumption, which may also be referred to as “feed”. Thus, as used herein, the term food product is used in a broad sense and may preferably be replaced by the term “food or feed product”. As the skilled person understands, it may not always be possible to strictly separate food and feed products in the context of the present description, since e.g. a liquid fraction of a product may be used for human consumption, while solid constituents of the same product, obtained e.g. by filtration or sedimentation, may be used as feed. Preferably, the liquid food product used for contacting is a non-enzymatically pretreated food product or is pretreated only with a cellulase and/or an amylase, i.e., preferably was pretreated with no other enzyme(s) than cellulase and/or amylase. More preferably, the liquid food product is a non-enzymatically pretreated food product; thus, the fructosyltransferase preferably is the first enzyme the liquid food product is contacted with; more preferably, the fructosyltransferase and optionally a pectinase is (are) the first enzyme(s) the liquid food product is contacted with, even more preferably, the fructosyltransferase and optionally a pectinase is (are) the only enzyme(s) the liquid food product is contacted with. For the avoidance of doubt, it is indicated that the term “food product” comprises end products, e.g. products ready for sale and/or consumption, as well as any intermediate products produced by a method described herein, which may be treated by further methods and/or may be used in the production of at least one further intermediate product or an end product. In view of the description elsewhere herein, the liquid food product used in the contacting step preferably is a crude product early in the production process and, thus, may e.g. a crude juice or mash obtained by crushing and/or pressing of fruit and/or vegetable, optionally pretreated by heat treatment and/or centrifugation. Preferably, the liquid food product used in the contacting step is a crude product early in the production process and, thus, may e.g. a crude juice or mash obtained by crushing and/or pressing of fruit and/or vegetable with no further pre-treatment.

The liquid food product comprises sucrose, preferably at a concentration of at least 1 g/L, more preferably at least 2 g/L, even more preferably at least 5 g/L, before said contacting. Thus, the liquid food product preferably is a plant-based food product, preferably a vegetable juice, vegetable puree, a fruit juice or a fruit puree. Preferably, the food product is a crude fruit or vegetable juice or a crude fruit or vegetable puree. The fruit juice or vegetable juice may be a clear or a cloudy fruit juice or vegetable juice; also, the fruit juice or vegetable juice may be a freshly prepared fruit juice or vegetable juice, a concentrated fruit juice or vegetable juice, may be prepared from a concentrated fruit juice or vegetable juice, or may be prepared from a non-concentrated fruit juice or vegetable juice. Preferably, the liquid food product is or comprises a juice or puree made from apple, orange, watermelon, grapefruit, plum, apricot, peach, mandarin, pineapple, lychee, mango, honey melon, banana, vegetables, onion, black root, celery, carrot, horseradish, red beet, and/or chestnut. The liquid food product may comprise components other than those specified herein above, in particular may comprise added sugar, e.g. sucrose, water, pH regulators, and/or other additives deemed appropriate by the skilled person. Also, the liquid food product may be a mixture of at least two of the aforesaid juices and/or purees, which mixture may also comprise further compounds as specified herein above In any case, however, the liquid food product shall be a liquid food product and shall comprise at least 1 g/L sucrose before said contacting. Preferably, the liquid food product is an orange juice, a pineapple juice, an apple juice, an apple mash, a banana puree, or an apricot puree.

Preferably, the liquid food product comprises sucrose at a concentration of less than 50 g/L, preferably less than 25 g/L, more preferably less than 20 g/L, still more preferably less than 15 g/L, even more preferably less than 10 g/L, most preferably less than 5 g/L, before said contacting. Thus, preferably the liquid food product is a low-sucrose food product before said contacting. Also preferably, after said contacting the residual sucrose concentration in said liquid food product is at most 50 g/L, preferably at most 25 g/L, more preferably at most 15 g/L, still more preferably at most 10 g/L, even more preferably at most 5 g/l. Thus, also preferably, the liquid food product is a low-sucrose food product after said contacting. As the skilled person understands, the sucrose concentration before contacting preferably is higher than the target sucrose concentration after the contacting; thus, in case contacting is performed until the sucrose concentration is at most 15 g/L, the sucrose concentration before the contacting preferably is at least 16 g/L, preferably at least 17 g/L, more preferably at least 20 g/L, still more preferably at least 25 g/L, most preferably at least 50 g/L. The above applies to other target concentrations mutatis mutandis.

The term “contacting” is understood by the skilled person. Preferably, the term relates to bringing a compound as specified into physical contact with a liquid food product and, thereby, e.g. allowing the liquid food product and the compound to interact. Thus, contacting preferably comprises preparing an admixture between the liquid food product and the fructosyltransferase.

Preferably, contacting is performed under conditions allowing enzymatic conversion of sucrose to at least one FOS by said fructosyltransferase. Said conditions comprise, preferably admixing the fructosyltransferase to said liquid food product, i.e. preferably using the water activity essentially as it is already present in said liquid food product. Said conditions preferably further comprise a time frame appropriate for the fructosyltransferase to be active; as the skilled person understands, said time frame may in particular depend on the amount of enzyme activity contacted; typical time frames may be of from 10 min to 24 h, preferably of from 15 min to 12 h, more preferably of from 20 min to 6 h, most preferably of from 25 min to 180 min. Said conditions preferably further comprise a temperature suitable for the fructosyltransferase to be active; as the skilled person understands, said temperature may in particular depend on the amount of enzyme activity contacted, but also on the sensitivity of the liquid food product to high temperatures and/or to temperature changes. Typical temperature ranges may e.g. be of from 0° C. to 75° C., preferably of form 4° C. to 60° C., more preferably of from 15° C. to 55° C., most preferably of from 18° C. to 55° C. Preferably, said conditions comprise contacting at a temperature of 20° C.±10° C. or 50° C.±10°. As the skilled person understands, the liquid food product may comprise one or more enzymes already present in the fruit or vegetable used to prepare the liquid food product; as referred to herein, such intrinsically present enzymes are not referred to by the term contacting, irrespective of which enzyme activity is concerned. Thus, the term contacting, as used herein, exclusively relates to contacting with an enzyme activity extraneously added to the liquid food product, preferably by addition of a composition of matter which is not a liquid food product as specified herein above.

The term “fructosyltransferase”, is understood by the skilled person to relate to an enzyme producing FOS under appropriate conditions by transferring fructose units to an appropriate acceptor. The fructosyltransferase preferably has sucrose:sucrose fructosyltransferase activity (E.C. 2.4.1.99); preferably, the fructosyltransferase has the aforesaid activity at low sucrose concentrations, preferably at a sucrose concentration of at most 15 g/L, preferably at most 10 g/L, more preferably at most 5 g/L. The fructosyltransferase may have one or more additional activity or activities, in particular invertase activity (sucrose hydrolase activity, E.C. 3.2.1.26).

Preferably, however, the fructosyltransferase activity is higher than the invertase activity, in particular at the aforesaid sucrose concentrations. Thus, preferably, at least 60%, preferably at least 70%, more preferably at least 80%, even more preferably at least 90%, even more preferably at least 95%, of the fructose comprised in sucrose in a solution comprising sucrose at a concentration of at most 5 g/L is converted to FOS by the fructosyltransferase. Thus, at least the aforesaid fraction of fructose comprised as fructose units in sucrose is converted to at least one FOS, the term “generating” fructose relating to generation of a fructose monomer which may, however, be an enzyme-bound intermediate of fructose which is then transferred to an appropriate acceptor molecule, e.g. sucrose. Thus, “generating” fructose, as referred to herein, does not necessarily comprise producing free fructose.

The fructosyltransferase is an Aspergillus niger fructosyltransferase. Fructosyltransferases from Aspergillus strains, such as A. niger, are known in the art, as described herein above.

Preferably, the fructosyltransferase comprises the amino acid sequence of the mature form of Genbank Acc. No. AHC54391.1 and/or the amino acid sequence of SEQ ID NO:1, or a sequence at least 80%, preferably at least 90%, more preferably at least 95%, still more preferably at least 98%, most preferably at least 99% identical thereto. More preferably, the fructosyltransferase comprises, preferably consists of, the amino acid sequence of the mature form of Genbank Acc. No. AHC54391.1 and/or the amino acid sequence of SEQ ID NO:1, which may e.g. be produced by expressing a gene encoding a sequence comprising the amino acid sequence of SEQ ID NO:2 in a suitable host cells, e.g. Trichoderma spec. cells. Preferably, the fructosyltransferase is produced by Trichoderma spec. cells, more preferably is purified from a Trichoderma spec. culture, most preferably a Trichoderma spec. culture supernatant, preferably as described in Berges et al. (1993), Curr Genet 24:53 or in WO 2018/009806 A1. The fructosyltransferase may be obtained as a crude or partially enriched extract from A. niger or Trichoderma spec. cells, or may be expressed recombinantly, e.g. in bacterial cells. The fructosyltransferase may, however, also be purified e.g. to at least 70%, preferably 80%, more preferably 90%, purity, e.g. by filtration, chromatography, precipitation, and/or any other purification step deemed appropriate by the skilled person.

The method for converting sucrose comprises a step of contacting the liquid food product with a fructosyltransferase under conditions allowing enzymatic conversion of sucrose to at least one FOS by said fructosyltransferase. Preferably, said contacting is performed at least until the sucrose concentration in said liquid food product is at most 15 g/L, preferably at most 10 g/L, more preferably at most 5 g/L; i.e. contacting is preferably continued until the aforesaid maximal sucrose concentration is reached or falls below said maximal sucrose concentration. Preferably, at least 60%, preferably at least 70%, more preferably at least 80%, even more preferably at least 90%, even more preferably at least 95%, of the fructose comprised in sucrose in the liquid food product before said contacting is converted to at least one FOS during said contacting. Thus, in view of the above, at least the aforesaid fractions of total fructose units liberated from sucrose are included in at least one FOS. Thus, preferably, less than 5 g/L, more preferably less than 2 g/L, fructose is produced by said converting. More preferably, the concentration of fructose in said liquid food product does not increase significantly during said contacting.

Preferably, the method described herein is an early step in the production of a liquid food product. Thus, the Aspergillus niger fructosyltransferase may in principle be added to the food product already before or during the initial crushing and/or pressing steps(s). Preferably, the Aspergillus niger fructosyltransferase is added directly after the aforesaid initial crushing and/or pressing step(s) and the contacting step is performed directly after said addition. Optionally, the crude preparation obtained in the initial crushing and/or pressing step(s) may be further pretreated by centrifugation. More preferably, the contacting step with the Aspergillus niger fructosyltransferase is not preceded by any other enzymatic pretreatment step; thus, the aforesaid contacting step preferably is the first enzymatic treatment step or is comprised in the first enzymatic treatment step of a liquid food product. Also preferably, the contacting step as referred to herein is the step preceding, preferably immediately preceding, an optional evaporation step, e.g. in manufacture of a juice concentrate or an optional ultrafiltration step, e.g. in apple juice production. Thus, the contacting step may preferably be performed intervening a centrifugation step and an evaporation step of a crude juice in a cloudy juice production process, a crushing step and a pressing step, and/or a first pressing step and a second pressing step in apple mash treatment, or a pressing step and an ultrafiltration step in apple juice production.

Optionally the method for converting sucrose further comprises contacting said food product with at least one pectinase. The term “pectinase” is known to the skilled person to relate to a group of hydrolytic enzymes degrading pectin, preferably having endo-polygalacturonase activity (EC 3.2.1.15), exopolygalacturonase (EC 3.2.1.67), arabinan endo-1,5-alpha-L-arabinanase activity (EC 3.2.1.99), arabinofuranosidase activity (EC 3.2.1.55), rhamnogalacturonan hydrolase (E.C. 3.2.1.171), pectin methyl esterase activity (EC 3.1.1.11), pectin acetyl esterase (E.C. 3.1.1.6) glucan 1,4-alpha-glucosidase activity (EC 3.2.1.3), pectate lyase (EC 4.2.2.2) and/or pectin lyase activity (EC 4.2.2.10). The contacting with at least one pectinase may be performed before, concomitant and/or after contacting with the fructosyltransferase, preferably is performed concomitantly to contacting with the fructosyltransferase. Preferably, the pectinase is an Aspergillus niger pectinase. Appropriate pectinases and blends thereof are commercially available, e.g. as ROHAPECT® PTE 100 (AB Enzymes), ROHAPECT® MA plus T (AB Enzymes), ROHAPECT® UF (AB Enzymes), Pectinex® Smash XXL (Novozymes), ROHAPECT® MA Plus HC (AB Enzymes), ROHAPECT® MA Plus (AB Enzymes), Pectinex® Yield Mash Extra (Novozymes), Pectinex® Yield Mash Plus (Novozymes), Pectinex® XXL (Novozymes), Pectinex® Ultra Clear (Novozymes), Pectinex® UF (Novozymes), Rapidase® Smart (DSM), Rapidase® Smart Clear (DSM), Rapidase® Power (DSM), Rapidase® Smart Plus (DSM), GAMYLOZYM® AFL (AB Enzymes), GAMMADEX® CAL (AB Enzymes), Hazyme® DCL (DSM), Amylase AG XXL (Novozymes), or Amylase AG 300 L (Novozymes). Preferably, ROHAPECT® PTE 100 and/or Pectinex® Smash XXL are used in orange juice treatment; ROHAPECT® MA plus T, ROHAPECT® MA Plus HC, ROHAPECT® MA Plus, Pectinex® Yield Mash Extra, Pectinex® Yield Mash Plus, Rapidase® Smart, and/or Rapidase® Smart Plus are used in apple mash treatment; GAMYLOZYM® AFL, ROHAPECT® UF; Pectinex® XXL; Pectinex® UF, Pectinex® Ultra Clear, Rapidase® Smart Clear, Rapidase® Power, GAMMADEX® CAL, Hazyme® DCL, Amylase AG XXL, and or Amylase AG 300 L are used in apple juice treatment; ROHAPECT® BiL, Pectinex® Ultra Tropical, Pectinex® Ultra SP-L, Rapidase® Pineapple, and or Pectinex® Power are used for pineapple juice treatment; ROHAPECT® PTE 100 and/or Pectinex® Smash XXL are used in banana or apricot purree treatment; all in combination with a fructosyltransferase as specified.

Optionally, the method for converting sucrose further comprises contacting, in particular pretreatment of, the liquid food product with at least one cellulase and/or amylase as specified herein above. Preferably, however, the method does not comprise enzymatic treatment steps preceding and/or following said contacting. Preferably, the method for converting sucrose further comprises decreasing turbidity and/or viscosity of the liquid food product, in particular in case said method further comprises contacting said food product with at least one pectinase.

Optionally, the method comprises a further step of inactivating said fructosyltransferase, e.g. by heat inactivation. Preferably, in such case, the liquid food product is heated to a temperature of at least 85°, more preferably at least 90° C., even more preferably at least 95° C., for at least 5 min, more preferably at least 10 min, most preferably at least 15 min. Preferably, heat inactivation is performed at a temperature of from 75° C. to 100° C., more preferably of from 80° C. to 97° C., most preferably of form 85° C. to 95° C., for one of the aforesaid time frames. Also optionally, the liquid food product is heat treated before said contacting, preferably as specified herein above.

After said contacting step, the liquid food product preferably has at least one altered property; as the skilled person understands, the term “altered” implies a comparison to an appropriate control, preferably a comparison to a an untreated or vehicle control, vehicle control meaning a control which was contacted to the carrier used to provide the respective enzyme(s), but not the enzyme(s). Preferably, the liquid food product has a decreased sucrose concentration and an increased FOS content after said contacting, preferably as specified herein above. Also preferably, the liquid food product has a decreased turbidity after said contacting, preferably compared to an untreated and/or a vehicle control. Methods of determining turbidity are known to the skilled person; preferably, turbidity is determined as Nephelometric Turbidity Unit (NTU) according to standard methods. Preferably, the liquid food product is centrifuged before turbidity measurement, more preferably at of from 1000 g to 10000 g, preferably 8000 g, for of from 1 to 30 min, preferably of from 2 to 15 min, more preferably for about 2 min. Also preferably, the liquid food product has a decreased viscosity, preferably a decreased dynamic viscosity, after said contacting, preferably compared to an untreated and/or a vehicle control.

Methods of determining viscosity in a liquid food product are known in the art. Preferably, dynamic viscosity is determined by rolling ball viscosimetry, preferably under standard condition, preferably at a temperature of 20° C.

Advantageously, it was found in the work underlying the present invention that Aspergillus niger fructosyltransferase (also referred to as “Aspergillus niger invertase”) has a high fructosyltransferase activity even at low sucrose concentrations; thus, the enzyme may be used to convert sucrose to FOSs essentially without producing free fructose; also, the enzyme may be used to convert sucrose to FOSs even at low sucrose concentrations, e.g. in food products low in sucrose and/or in cases where as complete as possible conversion of sucrose is desirable.

The definitions made above apply mutatis mutandis to the following. Additional definitions and explanations made further below also apply for all embodiments described in this specification mutatis mutandis.

The present invention further relates to a method for decreasing turbidity and/or viscosity of a liquid food product, said method comprising contacting said liquid food product with (i) a fructosyltransferase and (ii) a pectinase under conditions allowing said fructosyltransferase and said pectinase to be enzymatically active, wherein said contacting with (i) and (ii) preferably is concomitantly contacting.

The method for decreasing turbidity and/or viscosity may comprise steps in addition to those explicitly mentioned above. For example, further steps may relate, e.g., to heat treatment(s) as specified herein above, removal of solid constituents from the liquid food product, and the like.

Moreover, one or more of said steps may be assisted or performed by automated equipment.

Preferably, in addition to decreasing turbidity and/or viscosity of a liquid food product, the method also causes conversion of sucrose to at least one FOS, as specified herein above. The method may comprise further steps of enzymatic, chemical, and/or physical treatment; preferably, however, the method does not comprise enzymatic treatment steps preceding said contacting.

The present invention also relates to a composition comprising (i) an Aspergillus niger fructosyltransferase and (ii) a pectinase, wherein said Aspergillus niger fructosyltransferase preferably comprises an amino acid sequence as shown in SEQ ID NO:1 or a sequence at least 80%, preferably at least 90%, more preferably at least 95%, still more preferably at least 98%, most preferably at least 99% identical thereto.

The term “composition”, as referred to herein, relates to any composition of matter comprising the indicated components. Preferably, the composition is a solid composition, e.g. a lyophilisate or a granulate, which may e.g. be formulated with sodium chloride and/or maltodextrin. More preferably, the composition is a liquid composition, e.g. a stock solution, preferably ready for use for admixture to a liquid food product. The composition may comprise components in addition to the ones referred to herein, e.g. at least one solvent, in particular water; at least one stabilizer, in particular glycerol or sorbitol; at least one salt, e.g. a sodium salt, a potassium salt, a calcium salt, and/or a magnesium salt; at least one buffer, e.g. a phosphate or citrate buffer; at least one preservative, e.g. a benzoate or a sorbate; and/or one or more further additive(s) deemed appropriate by the skilled person. The composition may also comprise a filling agent, a moisture regulator, and/or an antioxidant, in particular in case the composition is a solid composition. As the skilled person understands, further components of the composition are preferably selected such as to not interfere with shelf life and/or activity of the fructosyltransferase and the pectinase. Also, further components of the composition are preferably selected such as to be compatible with use of the composition in a food product.

The present invention also relates to a kit comprising (i) an Aspergillus niger fructosyltransferase and (ii) a pectinase, wherein said Aspergillus niger fructosyltransferase preferably comprises an amino acid sequence as shown in SEQ ID NO:1 or a sequence at least 80%, preferably at least 90%, more preferably at least 95%, still more preferably at least 98%, most preferably at least 99% identical thereto.

The term “kit”, as used herein, refers to a collection of the aforementioned compounds, means or reagents, which are preferably packaged together, more preferably in a common housing.

The housing may be any kind of container and/or packaging deemed appropriate by the skilled person. The components of the kit may be comprised by separate containers (i.e. as a kit of separate parts) or provided in a single vial. Moreover, it is to be understood that the kit, preferably, is to be used for practicing the methods referred to elsewhere herein. It is, preferably, envisaged that all components are provided in a ready-to-use manner for practicing the methods referred to above. Further, the kit, preferably, contains instructions for carrying out said methods. The instructions can be provided by a user's manual in paper or electronic form. In addition, the manual may comprise instructions for administration and/or dosage instructions for carrying out the aforementioned methods using the kit of the present invention. Preferably, the kit comprises a diluent and/or a means of administration. Preferably, the fructosyltransferase and/or the pectinase are comprised in preparations comprising further components, preferably as described herein for compositions.

The present invention also relates to a use of an Aspergillus niger fructosyltransferase for converting sucrose into at least one fructooligosaccharide (FOS) in a liquid food product; to a use of an Aspergillus niger fructosyltransferase for producing at least one FOS in a liquid food product; to a use of an Aspergillus niger fructosyltransferase for decreasing the sucrose concentration in a liquid food product to at most 50 g/L, preferably at most 25 g/L, more preferably at most 15 g/L, still more preferably at most 10 g/L, even more preferably at most 5 g/l; and to a use of an Aspergillus niger fructosyltransferase and a pectinase for decreasing the turbidity and/or the viscosity of a liquid food product.

The aforesaid uses preferably comprise one or more features described herein above for the methods of the present invention. Preferably, the liquid food product is a low-sucrose food product. Preferably, the liquid food product is a low-sucrose food product before being made use of in one of the aforesaid uses, preferably comprising sucrose at a concentration of less than 50 g/L, preferably less than 25 g/L, more preferably less than 20 g/L, still more preferably less than 15 g/L, even more preferably less than 10 g/L, most preferably less than 5 g/L. more preferably, the liquid food product is a low-sucrose food product after being made use of in one of the aforesaid uses.

The present invention also relates to a food product obtained or obtainable by a method comprising the steps of a method of the present invention.

In view of the above, the skilled person understands that the food product may be a low-sucrose food product, may have a decreased turbidity and/or may have a decreased viscosity, all compared to the same type of food product not produced according to the present invention.

The food product comprises the fructosyltransferase and, optionally the pectinase, both as specified herein above. The aforesaid enzymes may still have the activities described herein above; more preferably, said enzymes no longer have the aforesaid activities; thus, said enzyme(s) may have been inactivated before or during production of the food product, e.g. by heat inactivation and/or may have been at least partially removed, e.g. using bentonite, active carbon, and/or ultrafiltration. Methods for identifying whether a food product comprises the aforesaid enzyme(s) are available, e.g. enzymatic tests for active enzymes, or e.g. immunological or mass spectrometry methods for inactive enzymes.

The food product may be a solid food product comprising as at least one component at least one liquid food product of the present invention. Preferably, the food product is a liquid food product as specified herein above, obtained or obtainable by a method comprising the steps of a method described herein above. Preferably, the food product is an orange juice, a pineapple juice, an apple juice, an apple mash, a banana puree, or an apricot puree.

The present invention also relates to a liquid food product comprising an Aspergillus niger fructosyltransferase and comprising sucrose at a concentration of less than 50 g/L, preferably less than 25 g/L, more preferably less than 20 g/L, still more preferably less than 15 g/L, even more preferably less than 10 g/L, most preferably less than 5 g/L.

To the liquid food product comprising an Aspergillus niger fructosyltransferase the above description of the food product obtained or obtainable by a method of the present invention applies mutatis mutandis. Preferably, the liquid food product is a fruit juice or a fruit puree, preferably is an orange juice, a pineapple juice, an apple juice, an apple mash, a banana puree, or an apricot puree. In view of the description herein above, the skilled person understands that the Aspergillus niger fructosyltransferase preferably is an added Aspergillus niger fructosyltransferase, i.e. is not intrinsically present in the liquid food product In view of the above, the following embodiments are particularly envisaged:

Embodiment 1: A method for converting sucrose comprised in a liquid food product into at least one fructooligosaccharide (FOS), the method comprising contacting said liquid food product with an Aspergillus niger fructosyltransferase under conditions allowing enzymatic conversion of sucrose to at least one FOS by said fructosyltransferase.

Embodiment 2: The method of embodiment 1, wherein said fructosyltransferase has fructosyltransferase activity at a sucrose concentration of at most 10 g/L, preferably at most 5 g/L.

Embodiment 3: The method of embodiment 1 or 2, wherein said liquid food product is a fruit juice, a fruit puree a vegetable juice, or vegetable puree.

Embodiment 4: The method of any one of embodiments 1 to 3, wherein said liquid food product is an orange juice, a pineapple juice, an apple juice, an apple mash, a banana puree, or an apricot puree.

Embodiment 5: The method of any one of embodiments 1 to 4, wherein said liquid food product is a low-sucrose food product before said contacting.

Embodiment 6: The method of any one of embodiments 1 to 5, wherein said liquid food product comprises sucrose at a concentration of at most at most 50 g/L, preferably at most 25 g/L, more preferably at most 20 g/L, still more preferably at most 15 g/L, even more preferably at most 10 g/L, most preferably at most 5 g/L, before said contacting.

Embodiment 7: The method of any one of embodiments 1 to 6, wherein said liquid food product is a non-enzymatically pretreated food product or, optionally, is enzymatically pretreated only with a cellulase and/or an amylase.

Embodiment 8: The method of any one of embodiments 1 to 7, wherein at least 60%, preferably at least 70%, more preferably at least 80%, even more preferably at least 90%, even more preferably at least 95%, of the fructose comprised in sucrose in the liquid food product before said contacting is converted to at least one FOS during said contacting.

Embodiment 9: The method of any one of embodiments 1 to 8, wherein contacting is performed at least until the sucrose concentration in said liquid food product is at most 15 g/L, preferably at most 10 g/L, more preferably at most 5 g/L; and/or wherein after contacting the residual sucrose concentration in said liquid food product is at most 15 g/L, preferably at most 10 g/L, more preferably at most 5 g/L.

Embodiment 10: The method of any one of embodiments 1 to 9 wherein less than 5 g/L, preferably less than 2 g/L, fructose is produced by said converting.

Embodiment 11: The method of any one of embodiments 1 to 10, wherein the concentration of fructose in said liquid food product does not increase significantly during said contacting.

Embodiment 12: The method of any one of embodiments 1 to 11, wherein said method further comprises contacting said food product with at least one pectinase.

Embodiment 13: The method of any one of embodiments 1 to 12, wherein said food product has a decreased turbidity after said contacting, preferably compared to an untreated and/or a vehicle control.

Embodiment 14: The method of any one of embodiments 1 to 13, wherein said liquid food product has a decreased viscosity after said contacting, preferably compared to an untreated and/or a vehicle control.

Embodiment 15: The method of any one of embodiments 1 to 14, wherein said method comprises contacting said food product with said fructosyltransferase and said pectinase concomitantly.

Embodiment 16: The method of any one of embodiments 1 to 15, wherein said liquid food product is a crude fruit juice or a crude fruit puree.

Embodiment 17: The method of any one of embodiments 1 to 16, wherein said method does not comprise enzymatic treatment steps preceding and/or following said contacting.

Embodiment 18: A method for decreasing turbidity and/or viscosity of a liquid food product, said method comprising contacting said liquid food product with (i) a fructosyltransferase and (ii) a pectinase under conditions allowing said fructosyltransferase and said pectinase to be enzymatically active.

Embodiment 19: The method of embodiment 18, wherein said contacting with said fructosyltransferase and said pectinase is concomitant contacting.

Embodiment 20: The method of embodiment 18 or 19, wherein said method does not comprise enzymatic treatment steps preceding said contacting.

Embodiment 21: The method of any one of embodiments 18 to 20, further comprising a feature of any one embodiments 1 to 17.

Embodiment 22: The method of any one of embodiments 1 to 21, wherein said fructosyltransferase comprises an amino acid sequence as shown in SEQ ID NO:1 or a sequence at least 80%, preferably at least 90%, more preferably at least 95%, still more preferably at least 98%, most preferably at least 99% identical thereto.

Embodiment 23: A composition comprising (i) a fructosyltransferase and (ii) a pectinase, wherein said fructosyltransferase comprises an amino acid sequence as shown in SEQ ID NO:1 or a sequence at least 80%, preferably at least 90%, more preferably at least 95%, still more preferably at least 98%, most preferably at least 99% identical thereto.

Embodiment 24: The composition of embodiment 23, wherein said composition is a liquid composition.

Embodiment 25: The composition of embodiment 23 or 24, further comprising at least one further compound selected from the list consisting of a solvent, a stabilizer, a salt, a buffer, and a preservative.

Embodiment 26: A kit comprising (i) a fructosyltransferase and (ii) a pectinase, wherein said fructosyltransferase comprises an amino acid sequence as shown in SEQ ID NO:1 or a sequence at least 80%, preferably at least 90%, more preferably at least 95%, still more preferably at least 98%, most preferably at least 99% identical thereto.

Embodiment 27: The kit of embodiment 26, wherein said components of the kit are comprised in a housing, preferably a common housing.

Embodiment 28: The method of any one of embodiments 12 to 22, the composition of any one of embodiments 23 to 25, or the kit of embodiment 26 or 27, wherein said pectinase comprises endo-polygalacturonase activity (EC 3.2.1.15), arabinan endo-1,5-alpha-L-arabinanase activity (EC 3.2.1.99), arabinofuranosidase activity (EC 3.2.1.55), pectin esterase activity (EC 3.1.1.11), glucan 1,4-alpha-glucosidase activity (EC 3.2.1.3), and/or pectin lyase activity (EC 4.2.2.10).

Embodiment 29: Use of an Aspergillus niger fructosyltransferase for converting sucrose into at least one fructooligosaccharide (FOS) in a liquid food product.

Embodiment 30: Use of an Aspergillus niger fructosyltransferase for producing at least one FOS in a liquid food product.

Embodiment 31: Use of an Aspergillus niger fructosyltransferase for decreasing the sucrose concentration in a liquid food product to at most 50 g/L, preferably at most 25 g/L, more preferably at most 15 g/L, still more preferably at most 10 g/L, even more preferably at most 5 g/l.

Embodiment 32: Use of a an Aspergillus niger fructosyltransferase and a pectinase for decreasing the turbidity and/or the viscosity of a liquid food product.

Embodiment 33: The use of any one of embodiments 29 to 32, wherein said Aspergillus niger fructosyltransferase comprises an amino acid sequence as shown in SEQ ID NO:1 or a sequence at least 80%, preferably at least 90%, more preferably at least 95%, still more preferably at least 98%, most preferably at least 99% identical thereto.

Embodiment 34: The use of any one of embodiments 29 to 33, wherein said liquid food product is a low-sucrose food product, preferably after conversion of sucrose into FOS, preferably comprising sucrose at a concentration of less than 50 g/L, preferably less than 25 g/L, more preferably less than 20 g/L, still more preferably less than 15 g/L, even more preferably less than 10 g/L, most preferably less than 5 g/L.

Embodiment 35: A food product obtained or obtainable by a method comprising the steps of a method of any one of embodiments 1 to 22 or 28.

Embodiment 36: The food product of embodiment 35, wherein said food product is a liquid food product.

Embodiment 37: The food product of embodiment 35 or 36, being an orange juice, an apple juice, a pineapple juice, an apple mash, a banana puree, or an apricot puree.

Embodiment 38: A liquid food product comprising an Aspergillus niger fructosyltransferase and comprising sucrose at a concentration of less than 50 g/L, preferably less than 25 g/L, more preferably less than 20 g/L, still more preferably less than 15 g/L, even more preferably less than 10 g/L, most preferably less than 5 g/L.

Embodiment 39: The liquid food product of embodiment 38, wherein said liquid food product is a fruit juice or a fruit puree, preferably is an orange juice, an apple juice, a pineapple juice, an apple mash, a banana puree, or an apricot puree.

Embodiment 40: The liquid food product of embodiment 38 or 39, wherein said Aspergillus niger fructosyltransferase is an added Aspergillus niger fructosyltransferase.

All references cited in this specification are herewith incorporated by reference with respect to their entire disclosure content and the disclosure content specifically mentioned in this specification.

### FIGURE LEGENDS

FIG. 1: Results of orange juice treatment; (A) and (B) concentrations of sugars over time with indicated treatments; (C) turbidity over time with indicated treatments; x-axes: time in min, FOS-E: FOSEnzyme preparation.

FIG. 2: Results of apple mash treatment; (A) concentrations of sugars after 60 min at 20° C.; (B) dynamic viscosity in [mPa*s], FOS-E: FOSEnzyme preparation.

FIG. 3: Results of apple juice treatment; concentrations of sugars after 90 min at 50° C., FOS-E: FOSEnzyme preparation.

FIG. 4: Results of pineapple juice treatment; concentrations of sugars over time with indicated treatments; x-axis: time in min, FOS-E: FOSEnzyme preparation.

FIG. 5: Results of banana puree treatment; (A) and (B) concentrations of sugars over time with indicated treatments; x-axis: time in min, FOS-E: FOSEnzyme preparation.

FIG. 6: Results of apricot puree treatment; (A) and (B) concentrations of sugars over time with indicated treatments; x-axis: time in min, FOS-E: FOSEnzyme preparation.

FIG. 7: HPAEC-PAD chromatogram showing signal intensity over elution time for extracts which were untreated (blank) or treated (20 ppm PTE100/1000 ppm FOSEnzyme product) as described in Example 5.

FIG. 8: Sugar and sc-FOS amount in orange juice in dependence on FOSEnzyme preparation dosage; (A) with 5 ppm of Rohapect PTE 100 added, (B) no additional enzyme.

FIG. 9: Sugar and sc-FOS amount in apple juice in dependence on FOSEnzyme preparation dosage. Additionally, 50 ppm of Rohapect MA PLUS T was added.

FIG. 10: Sugar and sc-FOS amount in banana puree in dependence on FOSEnzyme preparation dosage. Additionally, 5 ppm of Rohapect PTE 100 was added.

FIG. 11: Sugar and sc-FOS amount in carrot juice in dependence on FOSEnzyme preparation dosage. Additionally, 5 ppm of Rohapect PTE 100 was added.

The following Examples shall merely illustrate the invention. They shall not be construed, whatsoever, to limit the scope of the invention.

### EXAMPLE 1: ORANGE JUICE

substrate: Orange juice, direct juice from organic farming, REWE Bio pH value: 3.35 proceeding: Pour 50 ml orange juice into sealable Erlenmeyer flasks enzyme dosages: ROHAPECT® PTE 100 5 ppm (Aspergillus niger pectin lyase, EC 4.2.2.10, lot nr. R212243ST, specific activity 100 PTF/mg); all specific activities are as provided by the manufacturer.
FOSEnzyme product (Aspergillus niger fructosyltransferase (FOS-E), lot nr. R220627ST), 100, 500 & 1000 ppm; a control preparation lacking Aspergillus niger fructosyltransferase also lacked fructosyltransferase activity)
Reaction temperature: 50° C.
Response time: 120 minutes
Sampling after: 30 min-60 min-120 min
Samples were short-time exposed to 90° C. for 10 minutes
analytics: inactivated samples were frozen for (S/G/F measurement) test kit
Turbidity [NTU]: 1 ml centrifuge 13000 rpm for 2 min

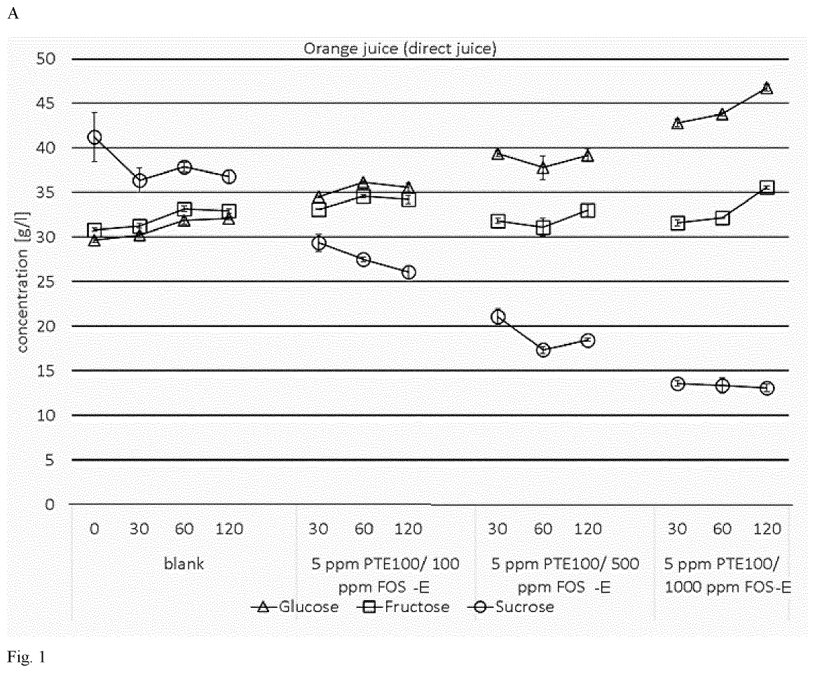

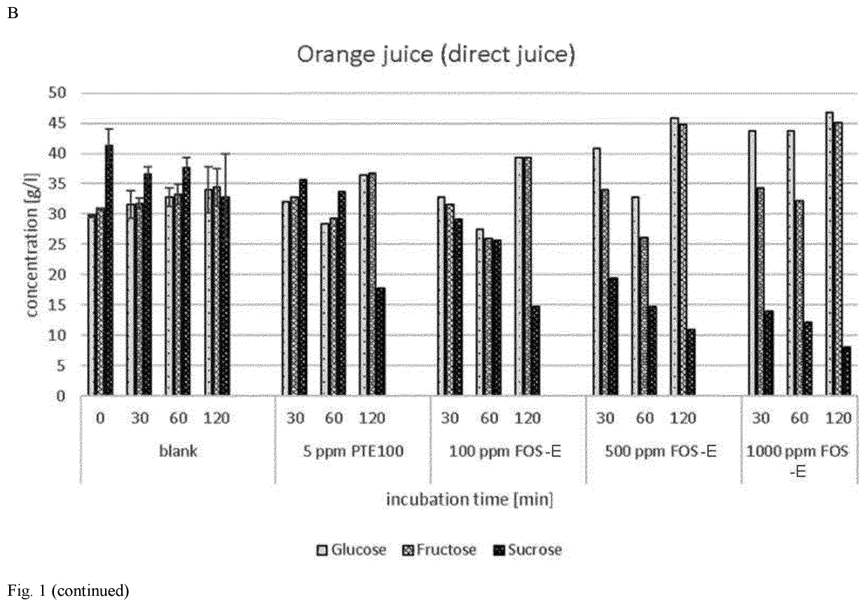

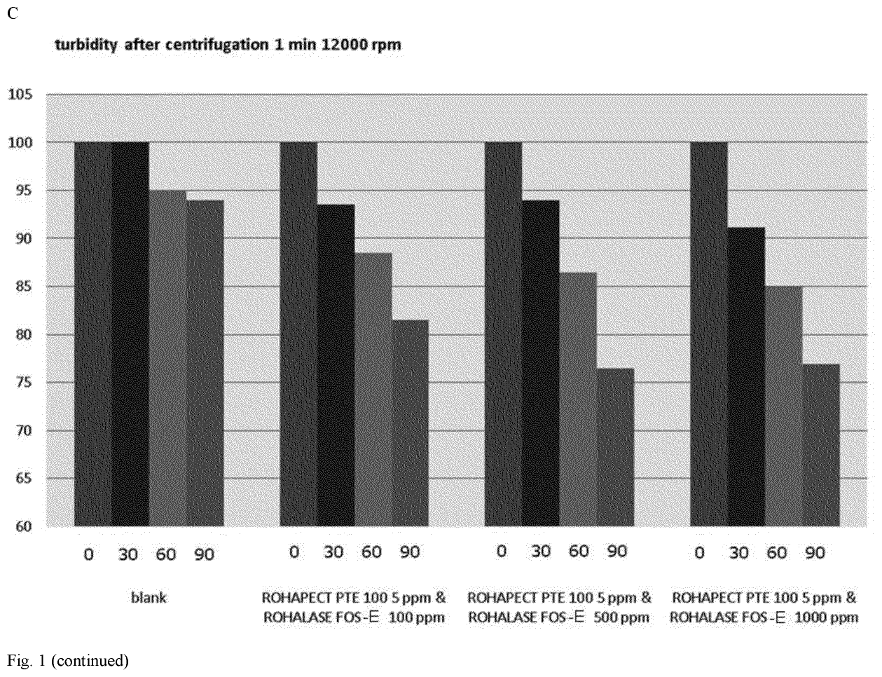

Results are shown in FIG. 1; cotreatment with fructosyltransferase (FOSEnzyme product) and pectinase (PTE100) causes a decrease of sucrose concentration with concomitant increase in glucose concentration (FIG. 1A) and a decrease in turbidity (FIG. 1C). The effect on sugar concentrations is mostly mediated by fructosyltransferase (FIG. 1B).

### EXAMPLE 2: APPLE MASH

substrate: Jonagold, pH 3.49
proceeding: apples were crushed in a meat grinder, 500 g mash was filled in a 1000 ml beaker enzyme dosages: ROHAPECT® MA plus T 100 ppm (pectinase blend comprising Endo-polygalacturonase (3.2.1.15, specific activity 1100 PGX/g) and Pectinesterase (3.1.1.11, specific activity 3150 PE/g), lot nr. R206179ST)
FOSEnzyme product 100 & 1000 ppm (R220627ST)
Reaction temperature: 20° C.
Response time: 60 minutes

After the reaction the apples mash was be pressed with a small Hafico Press at 0 bar 2 min-50 bar 2 min-100 bar 2 min-150 bar 2 min-200 bar 2 min analytics: inactivated samples were frozen for (S/G/F measurement) test kit Turbidity [NTU]: 1 ml centrifuge 13000 rpm for 2 min

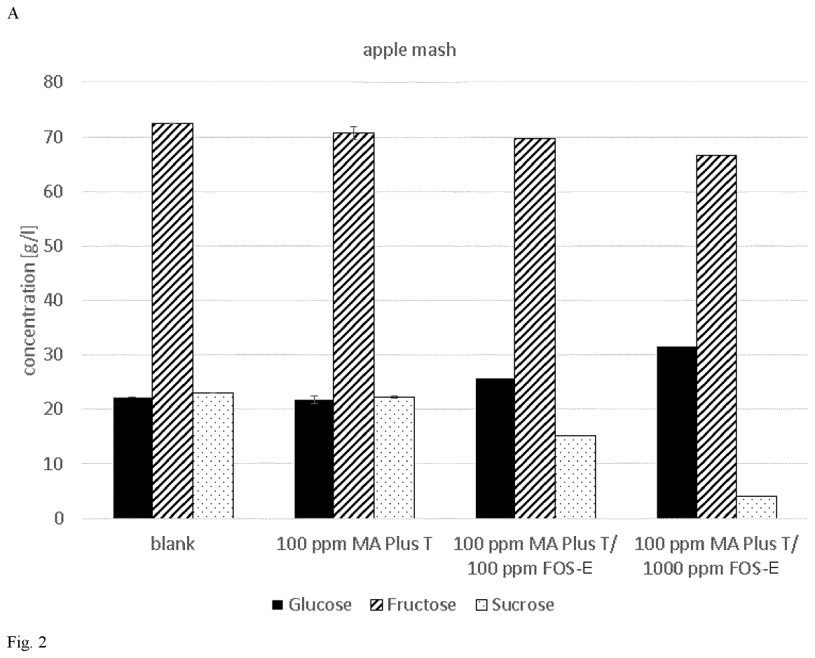

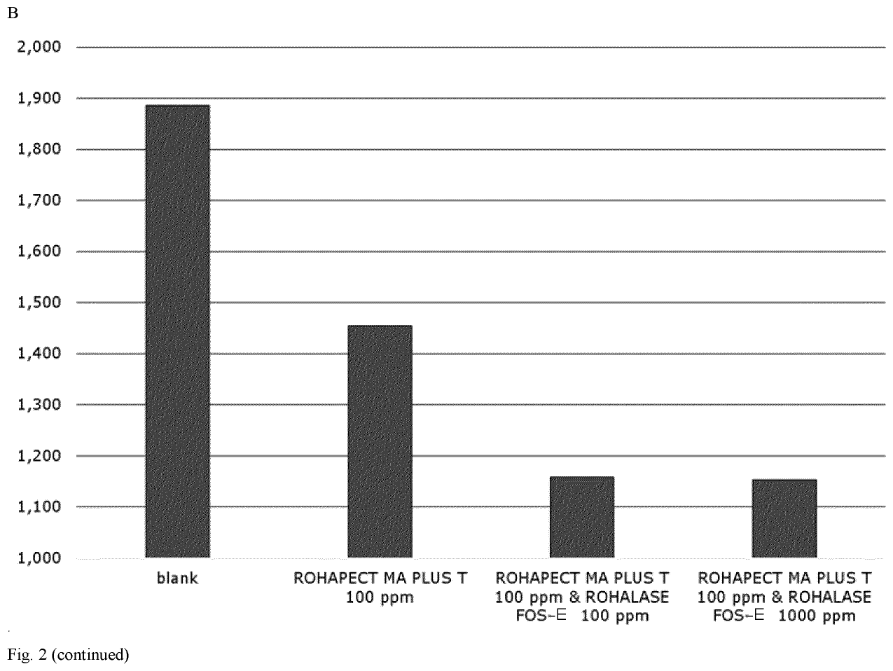

Results are shown in FIG. 2 and Table 1; cotreatment with fructosyltransferase (FOSEnzyme product) and pectinase (MA plus T) causes a decrease of sucrose concentration to less than 5 g/L with concomitant increase in glucose concentration (FIG. 2A) and a decrease in dynamic viscosity (FIG. 2B). Cotreatment has no negative effect on juice yield (Table 1), one of the most important parameters in apple juice manufacturing. Surprisingly an increase in juice turbidity (NTU) is observed in the juice pressed from a treated apple mash (Table 1), which is desirable in manufacturing cloudy apple juices. At the same time the viscosity of juice obtained by mash co-treatment is significantly reduced as compared to the treatment with ROHAPECT MA plus alone. This is a surprising effect as well and, following Darcy's law, this is leading to better filtration of the juice in the next process step and thus leads to an improved processing including better filtration in the following production steps in the industrial juice manufacturing plant.

### EXAMPLE 3: APPLE JUICE

substrate: Juice from Jonagold, pH 3.55
proceeding: See apple mash treatment (Example 2) with modified enzyme dosage enzyme dosage EL-2022/000031 30 ppm (pectinase blend comprising Endo-polygalacturonase (EC 3.2.1.15, specific activity 1250 PGX/g, Pectinesterase (EC 3.1.1.11, specific activity 1550 PE/g), Pectin lyase (EC 4.2.2.10, specific activity 30 PTF/mg), arabinan endo-1,5-alpha-L-arabinanase (EC 3.2.1.99, specific activity 100 ARAG/g, and glucan 1,4-alpha-glucosidase, 150 GAU/g)
FOSEnzyme product 100 & 500 ppm (lot nr. R220627ST)
GAMYLOZYM AFL 15 ppm
Reaction temperature: 50° C.; Response time: 120 minutes.
After the reaction 10 min 90° C., cool down up to 50° C.

An Amicon TCF 10-UF system is available. Membranes: SARTORIUS 11107 cellulose nitrate filter 0.2 μm were used.

Filtration parameter: Air pressure is 0.5 bar and. Time unit by weighing.

After Juice reaction time juice was directly transferred to the UF system and started. Temp. UF system 55° C.). After the reaction samples were heated (10 min 90° C.) and then cooled down to 50° C.
analytics: inactivated samples were frozen for (S/G/F measurement) test kit until use Turbidity [NTU]: 1 ml centrifuge 13000 rpm for 2 min

Results are shown in FIG. 3 and Table 8; cotreatment with fructosyltransferase (FOSEnzyme product) and pectinase (EL-2022/000031) causes a decrease of sucrose concentration to less than 5 g/L with concomitant increase in glucose concentration (FIG. 3). Cotreatment also unexpectedly led to significantly improved or higher flux rates during filtration, which means a significantly improved filtration performance in industrial manufacturing. Flux is described as the volume of juice filtered per m2 of membrane in 1 hr (Table 8).

### EXAMPLE 4: PINEAPPLE JUICE

substrate: pineapple, pH 3.4

proceeding: pineapples were ground in a meat grinder; mash was heated to 90° C. in the microwave (hot break) and then was directly transferred to a finisher (Alexander Werk).

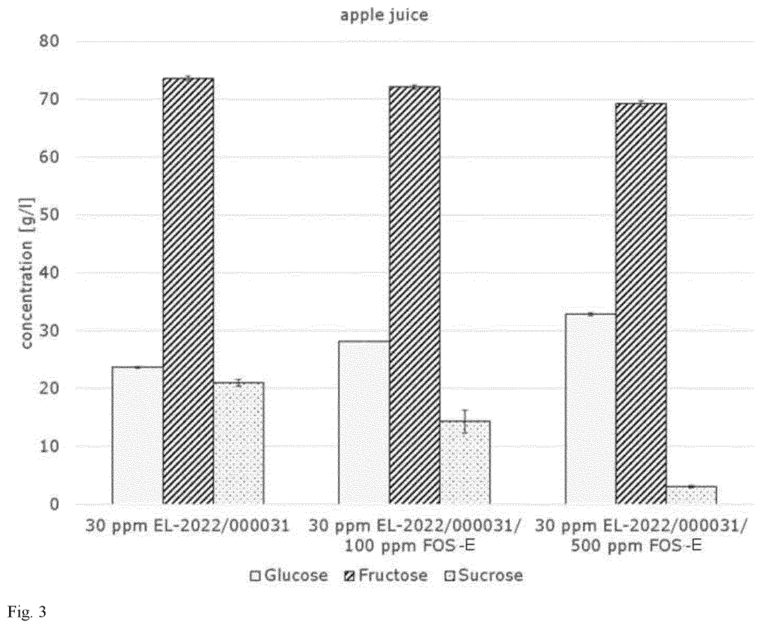

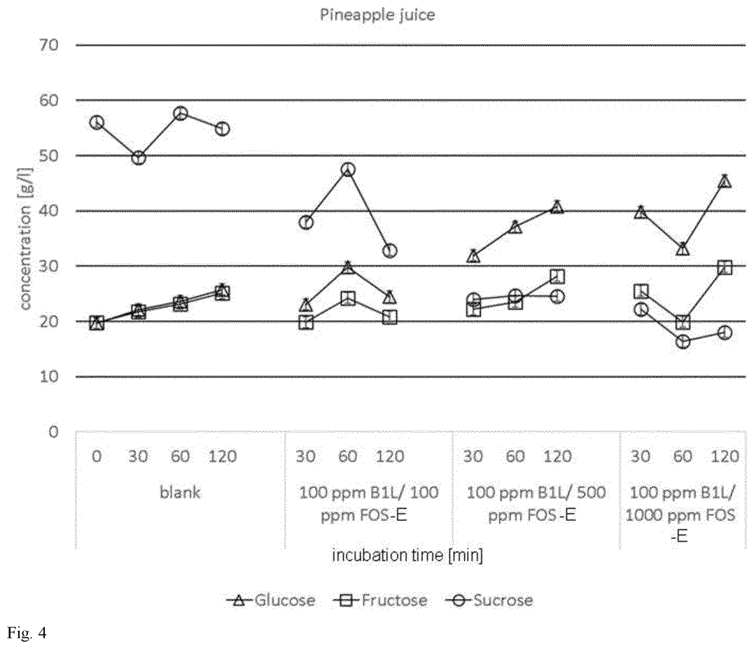

Juice enzyme dosage ROHAPECT® B1L 100 ppm (pectinase blend comprising Pectinesterase (EC 3.1.1.11, specific activity 450 PE/g) and Endo-polygalacturonase (EC 3.2.1.15, specific activity 400 PGX/g), as well as minor mannanase (EC 3.2.1.78) and cellulase (3.2.1.4) activities;
FOSEnzyme product 100, 500 & 1000 ppm
Reaction temperature: 50° C.
Response time: 120 minutes
Sampling after: 30 min-60 min-120 min
Samples were short-time exposed to 90° C. for 10 minutes
analytics: inactivated samples were frozen for (S/G/F measurement) test kit
Turbidity [NTU]: 1 ml centrifuge 13000 rpm for 2 min
Results are shown in FIG. 4; cotreatment with fructosyltransferase (FOSEnzyme product) and pectinase (B1L) causes a decrease of sucrose concentration with concomitant increase in glucose concentration.

### EXAMPLE 5: BANANA PUREE

substrate: banana, pH 4.75
proceeding: bananas were ground in a meat grinder
Mash was heated to 90° C. in the microwave to 90° C. (hot break) and then was directly transferred to a finisher (Alexander Werk), ascorbic acid was added to 250 mg/kg puree and the puree was frozen in 500 g portions until use
enzyme dosage ROHAPECT® PTE 100 20 ppm, lot nr. R212243ST, comprising pectin
lyase (EC 4.2.2.10, specific activity 100 PTF/mg)
FOSEnzyme product 100 & 1000 ppm, lot nr. R220627ST
Reaction temperature: 50° C.
Response time: 30 min/60 min
After the reaction time, the mash was inactivated at 90° C.
analytics: inactivated samples were frozen for (S/G/F measurement) test kit
Turbidity [NTU]: 1 ml centrifuge 13000 rpm for 2 min, viscosity from puree & supernatant The blank sample (60 min) and the enzyme treated sample (60 min; 20 ppm ROHAPECT® PTE 100/1000 ppm FOSEnzyme product) were analyzed by HPAEC-PAD (high-performance anion-exchange chromatography/pulsed amperometric detection) using a Dionex CarboPac PA1 column (Thermo Scientific). Standard substances (Glucose, Fructose, Sucrose, Kestose, Kestotetraose, Kestopentaose) were used for peak identification.

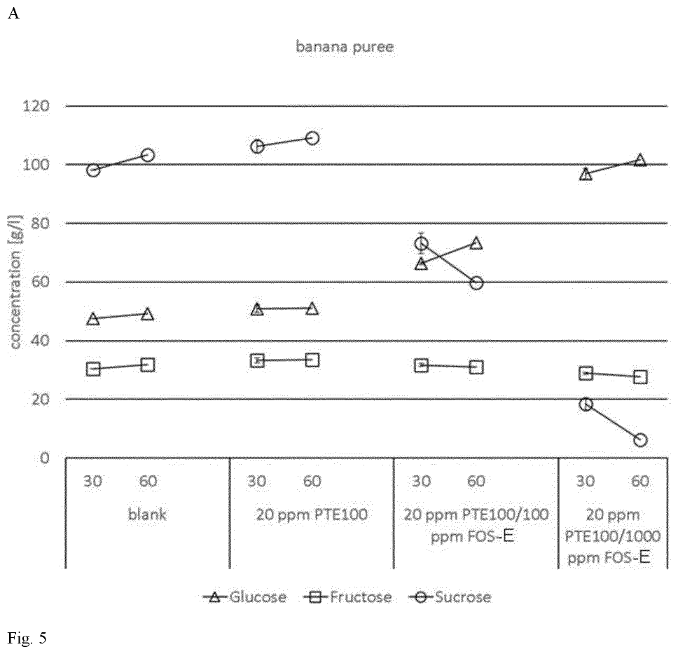

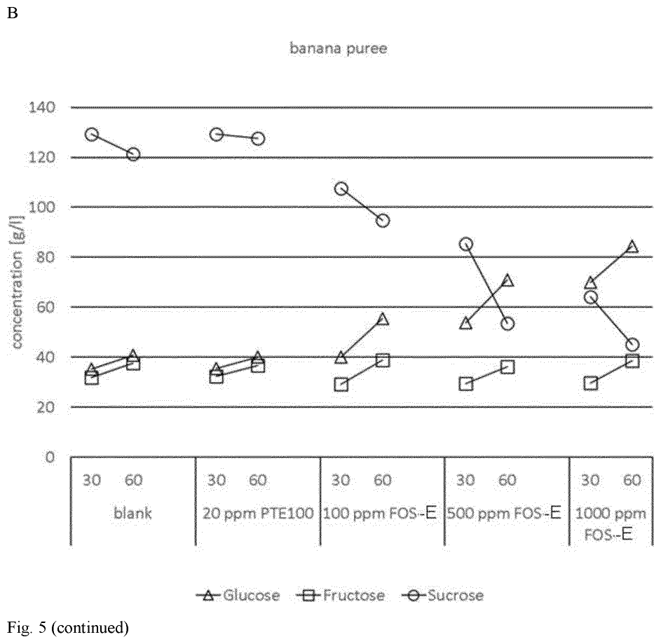

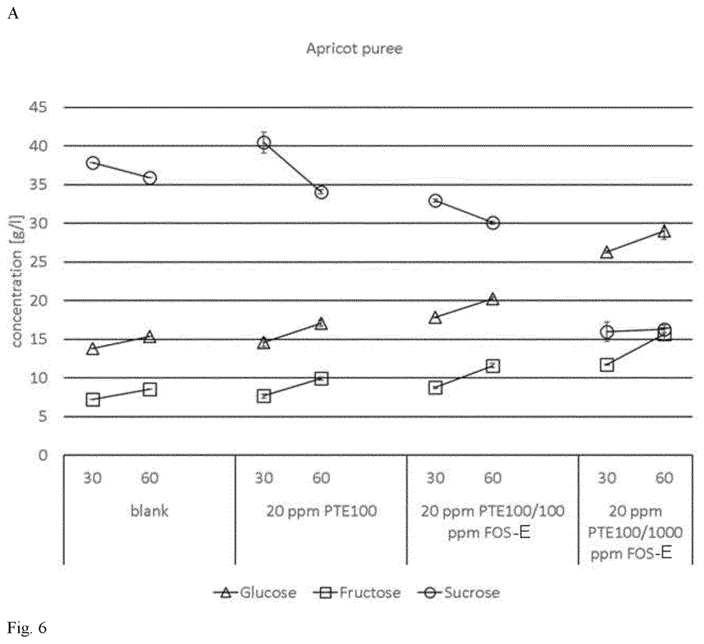

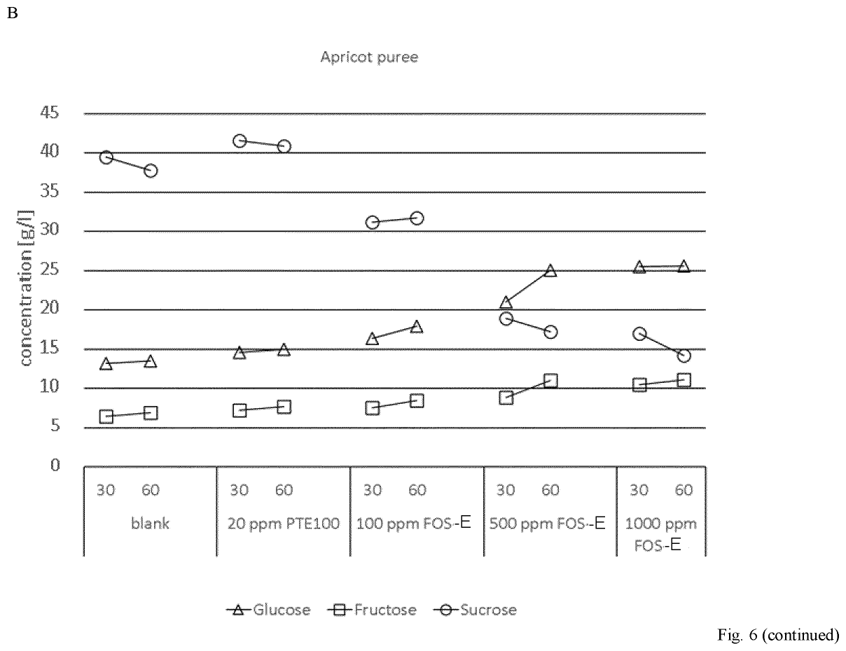

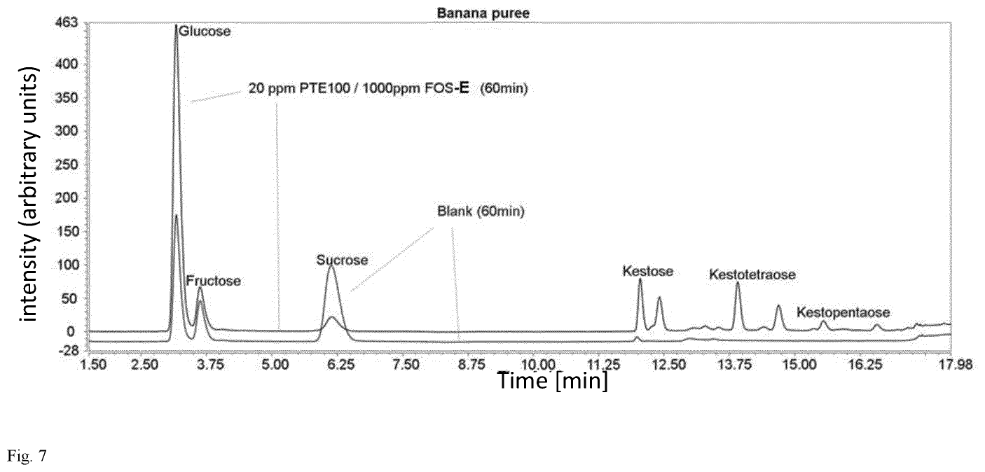

Results are shown in FIGS. 5 and 7 and Tables 2 to 4. As shown in FIG. 7, fructosyltransferase transferase treatment causes production of Glucose, Kestose, Kestotetraose, and Kestopentaose with a concomitant decrease in sucrose.

### EXAMPLE 6: APRICOT PUREE

Substrate: apricot, pH 3.55
proceeding: apricot were ground with a meat grinder; mash was heated to 90° C. in the microwave to 90° C. (hot break) and the mash then was directly transferred to a finisher (Alexander Werk); the puree was frozen in 500 g portions until use
enzyme dosage ROHAPECT® PTE 100 20 ppm
   FOSEnzyme product 100 & 1000 ppm
Reaction temperature: 50° C. Response time: 30 min/60 min
After the reaction time, the mash was inactivated at 90° C. analytics:
freeze inactivated samples for (S/G/F measurement) test kit
Turbidity [NTU]: 1 ml centrifuge 13000 rpm for 2 min, viscosity from puree & supernatant

### EXAMPLE 7: QUANTIFICATION OF FOS PRODUCTION

### General Method:

An Erlenmeyer flask containing the respective substrate solution as indicated herein below (orange juice, apple juice, banana puree, or carrot juice) was placed in a 50° C. pre-heated water bath. The substrate solution was stirred with a magnetic stirrer at 50 rpm. After temperature equilibration enzyme in dosages as indicated in the Figures was added. After 60 minutes incubation time samples were taken and heated at 90° C. for 10 minutes to inactivate the enzyme.

The amounts of sucrose (S), glucose (G), fructose (F), kestose (GF2), kestotetraose (GF3) and kestopentaose (GF4) in the samples were analysed by HPAEC (High Performance Anion Exchange Chromatography) with PAD (pulsed amperometric detection).

### EXAMPLE 7.1: ORANGE JUICE

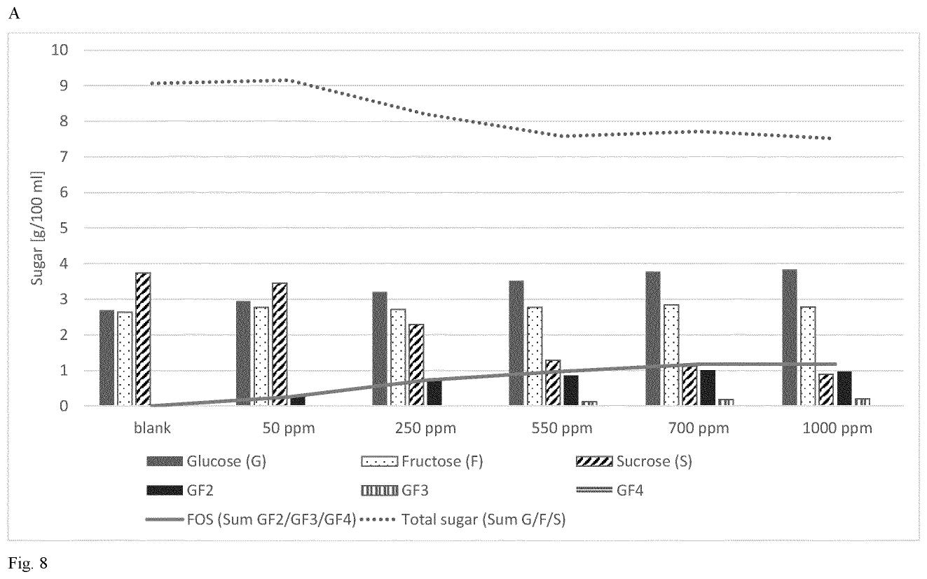

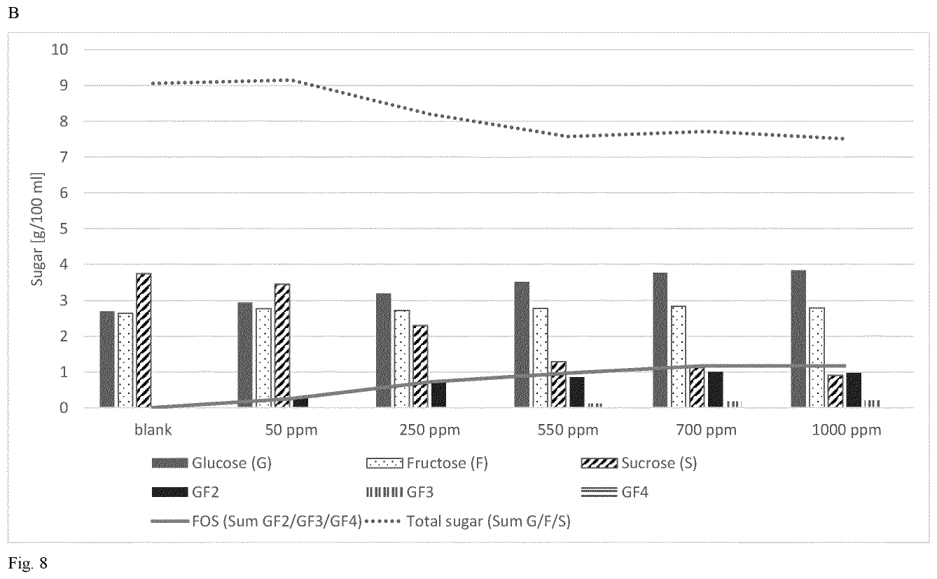

Orange juice was essentially obtained as indicated in Example 1; pH was 3.6. Incubation was with (FIG. 8A) or without (FIG. 8B) addition of 5 ppm of Rohapect PTE 100; Results are shown in FIG. 8.

### EXAMPLE 7.2: APPLE JUICE

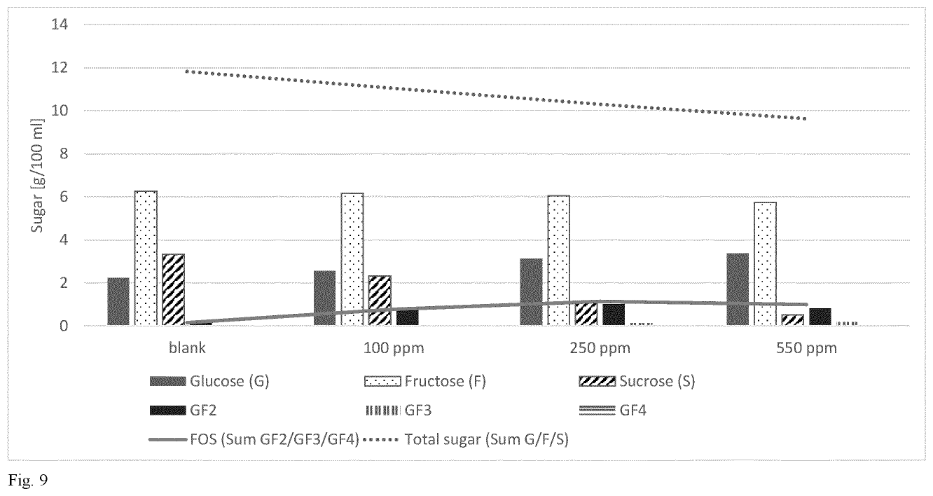

Apple juice was produced from crushed apples (apple mash) which was treated with enzymes at room temperature (˜22° C.) before pressing. After 60 minutes incubation time the apple mash was pressed to obtain the apple juice used in the Example; pH was 3.75. Incubation was in the additional presence of 50 ppm of Rohapect MA PLUS T; results are shown in FIG. 9.

### EXAMPLE 7.3: BANANA PUREE

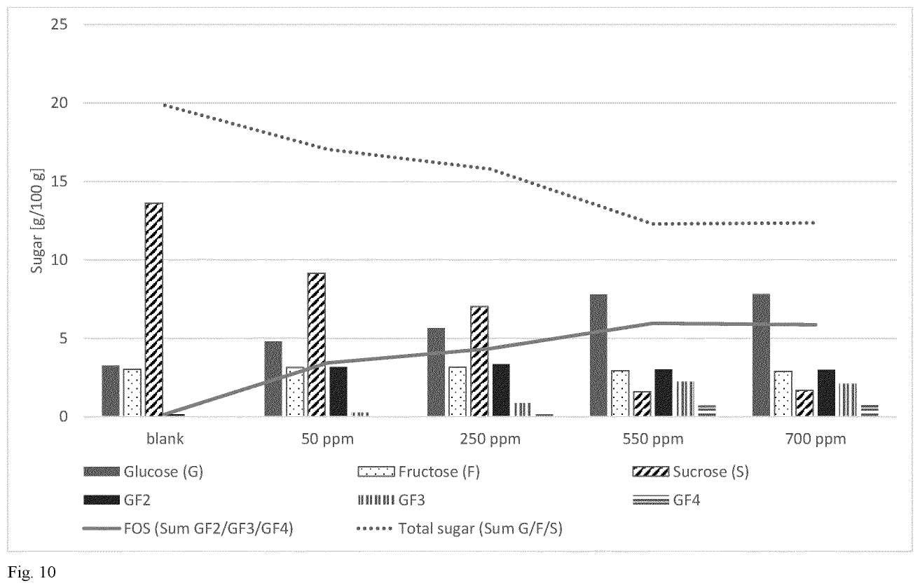

Banana puree was obtained essentially as indicated in Example 5; pH was 4.75. Incubation was in the presene of 5 ppm of Rohapect PTE 100; results are shown in FIG. 10.

### EXAMPLE 7.4: CARROT JUICE

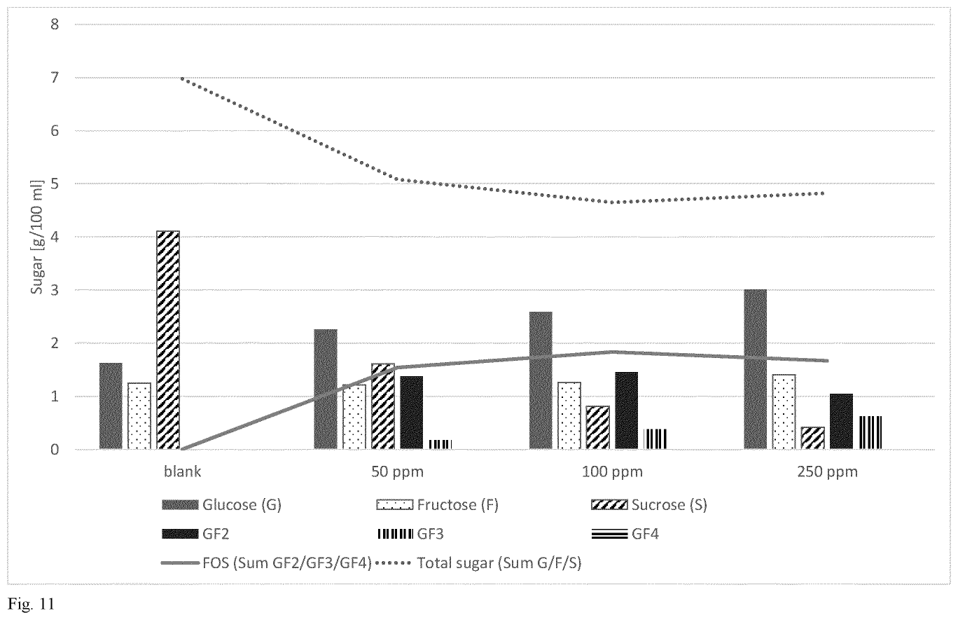

Carrot direct juice from organic farming, REWE Bio; pH was 5. Incubation was in the presence of 5 ppm of Rohapect PTE 100; results are shown in FIG. 11.

TABLE 1
apple mash treatment results

| | | | | | refractometer | | turbidity | | dyn. |
|---|---|---|---|---|---|---|---|---|---|
| | mash | Juice | Juice | | juice calculate | turbidity | [NTU] after | A-test | Viscosity |
| | detachment | [g] | [%] | ° Brix | 12°Brix [%] | [NTU] | centrif. | 1:1 | [mPa*s] |
| blank | + + + | 380 | 75.9 | 12.9 | 81.6 | 244 | 116 | pos | 1,886 |
| ROHAPECT ® MA PLUS T | + + + | 391 | 78.2 | 12.9 | 84.1 | 156 | 10.0 | pos | 1,455 |
| 100 ppm | | | | | | | | | |
| ROHAPECT ® MA PLUS T | + + + | 388 | 77.5 | 12.9 | 83.3 | 195 | 9.2 | pos | 1,158 |
| 100 ppm & FOSEnzyme | | | | | | | | | |
| product 100 ppm | | | | | | | | | |
| ROHAPECT ® MA PLUS T | + + + | 391 | 78.2 | 12.9 | 83.7 | 198 | 8.3 | pos | 1,153 |
| 100 ppm & FOSEnzyme | | | | | | | | | |
| product 1000 ppm | | | | | | | | | |

TABLE 2
Banana puree results, combined treatments.

| | rel. viscosity | turbidity [NTU] | | dyn. viscosity |
|---|---|---|---|---|
| | [%] Puree | after centrif. | A-test 1:1 | [mPa*s] |
| blank | 100 | 150 | pos | 20.050 |
| ROHAPECT PTE 100 20 ppm | 61.7 | 59 | pos | 1.379 |
| ROHAPECT PTE 100 20 ppm & | 53.3 | 42 | pos | 1.391 |
| FOSEnzyme product 100 ppm | | | | |
| ROHAPECT PTE 100 20 ppm & | 54.2 | 50 | pos | 1.383 |
| FOSEnzyme product 1000 ppm | | | | |

TABLE 3
Banana puree results, single treatments.

| | rel. viscosity | turbidity [NTU] | dyn. Viscosity |
|---|---|---|---|
| | [%] Puree | after centrif. | [mPa*s] |
| blank | 100 | 96 | 12.7 |
| ROHAPECT PTE 100 20 ppm | 64.8 | 36 | 1.4 |
| FOSEnzyme product 100 ppm | 88.1 | 133 | 14.0 |
| FOSEnzyme product 500 ppm | 88.6 | 115 | 12.3 |
| FOSEnzyme product 1000 ppm | 88.1 | 112 | 12.4 |

TABLE 4
Banana puree results, tastings

| | flavor |
|---|---|
| blank | Banana sweet |
| ROHAPECT PTE 100 20 ppm | Sweet; different consistency |
| FOSEnzyme product 100 ppm | less sweet than blank/less intense |
| FOSEnzyme product 500 ppm | less sweet than 100 ppm FOSEnzyme product/less intense |
| FOSEnzyme product 1000 ppm | less sweet than 500 ppm FOSEnzyme product/less intense |
| ROHAPECT PTE 100 20 ppm & | less sweet than blank and POHAPECT PTE 100/less intense |
| FOSEnzyme product 100 ppm | |
| ROHAPECT PTE 100 20 ppm & | less sweet than 100 ppm FOSEnzyme product/less intense |
| FOSEnzyme product 1000 ppm | |

TABLE 5
Apricot puree results, combined treatments.

| | rel. viscosity | turbidity [NTU] | | dyn. Viscosity |
|---|---|---|---|---|
| | [%] puree | after centrif. | A-test 1:1 | [mPa*s] |
| blank | 100 | 260 | pos | 20.169 |
| ROHAPECT PTE 100 20 ppm | 40.5 | 31 | pos | 4.775 |
| ROHAPECT PTE 100 20 ppm & | 41.7 | 32 | pos | 4.616 |
| FOSEnzyme product 100 ppm | | | | |
| ROHAPECT PTE 100 20 ppm & | 41.7 | 30 | pos | 4.616 |
| FOSEnzyme product 1000 ppm | | | | |

TABLE 6
Apricot puree results, single treatments.

| | rel. viscosity | turbidity [NTU] | dyn. Viscosity |
|---|---|---|---|
| | [%] puree | after centrif. | [mPa*s] |
| blank | 100 | 301 | 25.5 |
| ROHAPECT PTE 100 20 ppm | 57.6 | 22 | 1.2 |
| FOSEnzyme product 100 ppm | 90.9 | 256 | 22.7 |
| FOSEnzyme product 500 ppm | 95.5 | 278 | 21.1 |
| FOSEnzyme product 1000 ppm | 90.9 | 234 | 21.2 |

TABLE 7
Apricot puree results, tastings.

| | flavor |
|---|---|
| blank | “normal”/apricot slightly sour |
| ROHAPECT PTE 100 20 ppm | Scent different from blank/no apricot, sour |
| FOSEnzyme product 100 ppm | sweeter and rounder than blank/less intense |
| FOSEnzyme product 500 ppm | sweeter like 100 ppm FOSEnzyme product/less intense, less |
| | sour |
| FOSEnzyme product 1000 ppm | sweeter like 500 ppm FOSEnzyme product/less intense, less |
| | sour |
| ROHAPECT PTE 100 20 ppm & | sweet with a slight acidity |
| FOSEnzyme product 100 ppm | |
| ROHAPECT PTE 100 20 ppm & | less sweet, less acidity |
| FOSEnzyme product 1000 ppm | |

TABLE 8
Flux rates after treatment with enzymes:

| Filtration | | EL-2022/000031 30 ppm & | EL-2022/000031 30 ppm & |
|---|---|---|---|
| Time [min] | EL-2022/000031 30 ppm | FOSEnzyme product 100 ppm | FOSEnzyme product 500 ppm |
| 00:15:00 | 19.70 | 22.85 | 23.93 |
| 00:30:00 | 15.55 | 20.57 | 20.33 |

### LITERATURE CITED

Bergès et al. (1993), Curr Genet 24:53
Boddy et al. (1993), Curr Genet 24: 60
EP 1 951 885 A1
EP 2 294 093 A1
EP 3 231 293 A1
WO 2008/102336 A2
WO 2018/009806 A1
Yuan et al. (2006), Microbiology 152:3061

1-15. (canceled)

16. A method for converting sucrose comprised in a liquid food product into at least one fructooligosaccharide (FOS), the method comprising contacting said liquid food product with an Aspergillus niger fructosyltransferase under conditions allowing enzymatic conversion of sucrose to at least one FOS by said fructosyltransferase,
wherein the contacting is performed at least until the sucrose concentration in said liquid food product is at most 15 g/L.

17. The method of claim 16, wherein after contacting the residual sucrose concentration in said liquid food product is at most 10 g/L.

18. The method of claim 16, wherein at least 60% of the fructose comprised in sucrose in the liquid food product before said contacting is converted to at least one FOS during said contacting.

19. The method of claim 16, wherein less than 5 g/L fructose is produced by said converting.

20. The method of claim 16, wherein the concentration of fructose in said liquid food product does not increase significantly during said contacting.

21. The method of claim 16, wherein said liquid food product is a non-enzymatically pretreated food product or is pretreated only with a cellulase and/or an amylase.

22. The method of claim 16, wherein said liquid food product is a low-sucrose food product comprising sucrose at a concentration of less than 50 g/L before said contacting.

23. The method of claim 16, wherein said method further comprises contacting said food product with at least one pectinase.

24. The method of claim 16, wherein said food product has a decreased turbidity and/or viscosity after said contacting compared to an untreated and/or a vehicle control.

25. A method for decreasing the sucrose concentration in a liquid food product to at most 15 g/L comprising converting sucrose comprised in said liquid food product into at least one fructooligosaccharide (FOS) according to the method of claim 16.

26. The method of claim 16, wherein said Aspergillus niger fructosyltransferase comprises an amino acid sequence as shown in SEQ ID NO:1 or a sequence at least 80% identical thereto.

27. The method of claim 16, wherein said liquid food product is a fruit juice, a fruit puree, a vegetable juice, or vegetable puree.

28. The method of claim 16, wherein said liquid food product is an orange juice, an apple juice, a pineapple juice, an apple juice, an apple mash, a banana puree, or an apricot puree.

29. A liquid food product obtained or obtainable by a method comprising the steps of the method of claim 16.

30. A liquid food product comprising an Aspergillus niger fructosyltransferase and comprising sucrose at a concentration of at most 15 g/L.

31. The liquid food product of claim 30, wherein said Aspergillus niger fructosyltransferase comprises an amino acid sequence as shown in SEQ ID NO:1 or a sequence at least 80% identical thereto,

32. The liquid food product of claim 30, wherein said liquid food product is a fruit juice, a fruit puree, a vegetable juice, or a vegetable puree.

33. The liquid food product of claim 30, wherein said liquid food product is an orange juice, an apple juice, a pineapple juice, an apple juice, an apple mash, a banana puree, or an apricot puree.

34. The liquid food product of claim 30, obtained or obtainable by a method comprising contacting a liquid food product with an Aspergillus niger fructosyltransferase under conditions allowing enzymatic conversion of sucrose to at least one fructooligosaccharide (FOS) by said fructosyltransferase,
wherein the contacting is performed at least until the sucrose concentration in said liquid food product is at most 15 g/L.


SEQUENCE LISTING (2 sequences)

SEQ ID NO: 1 - protein, 604 aa, Aspergillus niger
SVVIDYNVAPPNLSTLPNGSLFETWRPRAHVLPPNGQIGDPCLHYTDPSTGLFHVGFLHDGSGISSATTDDLATYKDLNQGNQVIVPGGINDPVAVFDGSVIPSGINGLPTLLYTSVSFLPIHWSIPYTRGSETQSLAVSSDGGSNFTKLDQGPVIPGPPFAYNVTAFRDPYVFQNPTLDSLLHSKNNTWYTVISGGLHGKGPAQFLYRQYDPDFQYWEFLGQWWHEPTNSTWGNGTWAGRWAFNFETGNVFSLDEYGYNPHGQIFSTIGTEGSDQPVVPQLTSIHDMLWVSGNVSRNGSVSFTPNMAGFLDWGFSSYAAAGKVLPSTSLPSTKSGAPDRFISYVWLSGDLFEQAEGFPTNQQNWTGTLLLPRELRVLYIPNVVDNALARESGASWQVVSSDSSAGTVELQTLGISIARETKAALLSGTSFTESDRTLNSSGVVPFKRSPSEKFFVLSAQLSFPASARGSGLKSGFQILSSELESTTVYYQFSNESIIVDRSNTSAAARTTDGIDSSAEAGKLRLFDVLNGGEQAIETLDLTLVVDNSVLEIYANGRFALSTWVRSWYANSTNISFFQNGVGGVAFSNVTVSEGLYDAWPDRQS

SEQ ID NO: 2 - protein, 628 aa, Aspergillus niger
MKLQTASVLLGSAAAASPSMQTRASVVIDYNVAPPNLSTLPNGSLFETWRPRAHVLPPNGQIGDPCLHYTDPATGLFHVGFLHDGSGISSATTDDLATYQDLNQGNQVIVPGGINDPVAVFDGSVIPNGINGLPTLLYTSVSFLPIHWSIPYTRGSETQSLAVSSDGGSNFTKLDQGPVIPGPPFAYNVTAFRDPYVFQNPTLDSLLHSKNNTWYTVISGGLHEKGPAQFLYRQYDSDFQYWEYLGQWWHEPTNSTWGNGTWAGRWAFNFETGNVFSLDEYGYNPHGQIFTTIGTEGSDLPVVPQLTSIHDMLWVSGTVSRNGSVSFTPNMAGFLDWGFSSYAAAGKVLPSTSLPSTKSGAPDRFISYVWLSGDLFEQAEGFPTNQQNWTGTLLLPRELRVLYIPNVVDNALARESGASWQVVSSDSSAGTVELQTLGISIARETKAALLSGTSFTESDRTLNSSGVVPFKRSPSEKFFVLSAQLSFPASARGSGLKSGFQILSSEHESTTVYYQFSNESIIVDRSNTSAAARTTDGIDSSAEAGKLRLFDVLNDGEQAIETLDLTLVVDNSVLEVYANGRFALSTWVRSWYANSTNISFFHNGVGGVAFSNVTVSEGLYDAWPDRQS